(19) United States
(12) Patent Application Publication
(10) Pub. No.: US 2026/0107895 A1
(43) Pub. Date: Apr. 23, 2026

(54) MELON VARIETIES 'ML3627' AND 'ML3326'

(71) Applicant: Enza Zaden Beheer B.V., Enkhuizen (NL)

(72) Inventor: Dominique Marie Michel Chambeyron, Gordes (FR)

(21) Appl. No.: 19/339,636

(22) Filed: Sep. 25, 2025

Related U.S. Application Data
(60) Provisional application No. 63/699,354, filed on Sep. 26, 2024.

(51) Int. Cl. A01H 6/34; A01H 5/08

(52) U.S. Cl. CPC A01H 6/344; A01H 5/08

(57) ABSTRACT

New melon varieties designated ‘ML3627’ and ‘ML3326’ are described.

### CROSS-REFERENCE TO RELATED APPLICATIONS

This application claims the benefit of priority to U.S. Provisional Application No. 63/699,354, filed on Sep. 26, 2024, the entire content of which is hereby incorporated by reference.

### FIELD

The present disclosure relates to plants, plant parts, and plant breeding. The present disclosure includes new melon, Cucumis melo L., varieties designated ‘ML3627’ and ‘ML3326’.

### BACKGROUND

Cultivated forms of melon belong to the diploid species Cucumis melo L. of the Cucurbitaceae family. Cultivated melons grow as annual plants with large, broad leaves. Leaves are simple, either three-or five-lobed, and borne singly at the nodes, with a great variation of size, color, and shape among cultivars. The flowers can be monoecious, producing both male and female flowers, or andromonoecious, producing self-pollinated and male flowers on the same plant. Fruits (pepo) may be round, oblong, or elliptical with rind colors varying from white, green, to yellow and rind patterning varying widely. The flesh of cultivated melons can have a variety of different colors, e.g., orange, light green, or white. Seeds can be ovate to oblong, and can be yellowish or white in color. Fresh melons are consumed in many forms, generally fresh, sliced, or as an ingredient in prepared foods.

There are six horticultural groupings of C. melo: Cantalupensis (e.g., cantaloupe, muskmelon), Inodorus (e.g., winter melon), Flexuosus (e.g., snake melon), Conomom (e.g., pickling melon), Dudaim (e.g., pomegranate melon), and Momordica (e.g., snap melon). The Cantalupensis group is the most economically important for commercial production around the world. Common types of this group include: ‘Persian’ (large fruit with heavy netting), ‘Charentais’ (thin rinds and only sparse netting, popular in Europe and Africa), ‘Harper’ (tolerant to detrimental conditions, light tan to green, firm, and tough rind, covered in a rough, raised, and light brown exterior) and ‘Galia’ (green-fleshed netted melons).

Melon is an important and valuable field crop with respect to which melon consumers and/or melon growers consider characteristics such as flesh taste (e.g., sweetness), flesh color, flesh texture, fruit size, fruit shape, fruit rind pattern, fruit shelf life, fruit yield, disease resistance, pest (e.g., insect) resistance, drought tolerance, flood tolerance, heat tolerance, cold tolerance, herbicide resistance, modified fatty acid metabolism, modified carbohydrate metabolism, nutritional quality, sex determination, male sterility, harvestability, and/or industrial utility. Melon fruit comes in a wide range of sizes, from as small as one kilogram up to twenty or more kilograms. There are dozens of rind patterns possible on melon fruit. Melon fruit can have an array of shapes from round to oval (length/width ratios up to 3:1).

Disease resistance characteristics can contribute to the yield and quality of fruit and flesh. Examples of diseases that can impact melon yield and quality include, without limitation, powdery mildew, cucumber mosaic virus (CMV), cucurbit yellow stunting disorder virus (CYSDV) virus, downy mildew, fusarium wilt, gummy stem blight, and phytophthora rot.

### SUMMARY

Melon is an important and valuable field crop. Thus, there is a continued need for new melon varieties that are appealing to consumers and agronomically sound. The present disclosure provides, among other things, plants, plant parts, and uses of melon varieties with advantageous characteristics (e.g., flesh taste (e.g., sweetness), flesh color, flesh texture, fruit size, fruit shape, fruit rind pattern, fruit shelf life, fruit yield, disease resistance, pest (e.g., insect) resistance, drought tolerance, flood tolerance, heat tolerance, cold tolerance, herbicide resistance, modified fatty acid metabolism, modified carbohydrate metabolism, nutritional quality, sex determination, male sterility, harvestability, and/or industrial utility), which varieties are referred to herein as melon varieties (or melon cultivars) ‘ML3627’ and ‘ML3326’.

The present disclosure provides, among other things, a melon (Cucumis melo L.) variety seed designated as ‘ML3627’ and having NCIMB Accession Number X1 (representative seed of said melon variety ‘ML3627’ having been deposited under NCIMB Accession Number X1).

In some embodiment, the present disclosure provides a Cucumis melo L. melon plant produced by growing ‘ML3627’ melon seed and/or parts isolated therefrom (which plants and parts can be referred to, e.g., as ‘ML3627’ plants and ‘ML3627’ parts, respectively). In some embodiments, the present disclosure provides an ‘ML3627’ Cucumis melo L. seed and/or a seed having all, or essentially all, of the physiological and morphological characteristics of an ‘ML3627’ Cucumis melo L. seed. In some embodiments, the present disclosure provides plants grown from such seeds and/or parts isolated therefrom, such plants optionally having all, or essentially all, of the physiological and morphological characteristics of a Cucumis melo L. plant produced by growing ‘ML3627’ melon seed having NCIMB Accession Number X.

Melon plant parts include leaves, ovules, pollen grains, seeds, scions, rootstocks, fruits, parts of fruits, flowers, roots, root tips, stems, hypocotyls, petioles, cotyledons, cuttings, anthers, pistils, stalks, meristems, protoplasts, cells, and/or the like, and portions thereof. In some embodiment, the present disclosure provides leaves, ovules, pollen grains, seeds, scions, rootstocks, fruits, parts of fruits, flowers, roots, root tips, stems, hypocotyls, petioles, cotyledons, cuttings, anthers, pistils, stalks, meristems, protoplasts, cells, and/or the like, and portions thereof, isolated from ‘ML3627’ melon plants. In certain embodiments, the present disclosure provides pollen grains or ovules isolated from ‘ML3627’ melon plants. In some embodiment, the present disclosure provides protoplasts produced from ‘ML3627’ melon plants or parts. In some embodiment, the present disclosure provides tissue and/or culture produced from protoplasts or regenerable cells of and/or derived from ‘ML3627’ melon plants. In some embodiment, said protoplasts or regenerable cells are produced from a plant part selected from the group consisting of root, root tip, meristematic cell, stem, hypocotyl, petiole, cotyledon, leaf, flower, anther, pollen, pistil, stalk, and fruit. The present disclosure provides melon plants regenerated from tissue culture, where the plants regenerated from the tissue culture have all, or essentially all, of the physiological and morphological characteristics of ‘ML3627’ melon.

In some embodiment, the present disclosure provides a method of producing a seed of a ‘ML3627’-derived melon plant, including the steps: crossing a melon designated as ‘ML3627’, representative sample of seed having been deposited under NCIMB Accession Number X1, with itself or a second melon plant; and whereby seed of a ‘ML3627’-derived melon plant forms. In some embodiment, the method further includes (a) crossing a plant grown from ‘ML3627’-derived melon seed with itself or a second melon plant to yield additional ‘ML3627’-derived melon seed; (b) growing the additional ‘ML3627’-derived melon seed of step (a) to yield additional ‘ML3627’-derived melon plants; and (c) repeating the crossing and growing of steps (a) and (b) for at least one additional generation (e.g., an additional 3-10 generations) to generate further ‘ML3627’-derived melon plants. In some embodiment, the present disclosure further provides melon plants, melon parts from the melon plants, and seeds produced therefrom where the melon plant is grown from seed produced by the methods of any of the above embodiments. In some embodiment, the present disclosure provides methods of vegetatively propagating a plant of melon variety ‘ML3627’, the method including the steps of: (a) collecting a tissue capable of being propagated from a plant of melon variety ‘ML3627’; and (b) producing a rooted plant from said tissue.

The present disclosure provides, among other things, a melon plant designated ‘ML3627’. The present disclosure thus relates to the plants of melon ‘ML3627’, the plant parts of melon ‘ML3627’, and to the seeds of melon ‘ML3627’. The present disclosure also relates to methods for producing other melon cultivars or hybrids derived from melon ‘ML3627’ and to the melon cultivars and hybrids derived by the use of those methods.

In some embodiment, the present disclosure provides a method of producing a plant of melon variety ‘ML3627’ comprising an added desired trait (e.g., a trait that is advantageous for commercialization, production, and/or marketability), the method comprising introducing a transgene conferring a desired trait into a plant of melon variety ‘ML3627’.

In some embodiment, the present disclosure provides single gene converted plants of melon ‘ML3627’. The single transferred gene may preferably be a dominant or recessive allele. In various embodiments, the single transferred gene can confer a trait such as sex determination, herbicide resistance, insect resistance, resistance for bacterial, fungal, or viral disease, improved harvest characteristics, enhanced nutritional quality, and/or improved agronomic quality. The single gene may be a naturally occurring melon gene or a transgene introduced through genetic engineering techniques.

The present disclosure provides, among other things, a melon (Cucumis melo L.) variety seed designated as ‘ML3326’ and having NCIMB Accession Number X2 (representative seed of said melon variety ‘ML3326’ having been deposited under NCIMB Accession Number X2).

In some embodiment, the present disclosure provides a Cucumis melo L. melon plant produced by growing ‘ML3326’ melon seed and/or parts isolated therefrom (which plants and parts can be referred to, e.g., as ‘ML3326’ plants and ‘ML3326’ parts, respectively). In some embodiments, the present disclosure provides an ‘ML3326’ Cucumis melo L. seed, and/or a seed having all, or essentially all, of the physiological and morphological characteristics of an ‘ML3326’ Cucumis melo L. seed. In some embodiments, the present disclosure provides plants grown from such seeds and/or parts isolated therefrom, such plants optionally having all, or essentially all, of the physiological and morphological characteristics of a Cucumis melo L. plant produced by growing ‘ML3326’ melon seed having NCIMB Accession Number X2.

Melon plant parts include melon leaves, ovules, pollen grains, seeds, scions, rootstocks, fruits, parts of fruits, flowers, roots, root tips, stems, hypocotyls, petioles, cotyledons, cuttings, anthers, pistils, stalks, meristems, protoplasts, cells, and/or the like, and portions thereof. In some embodiment, the present disclosure provides leaves, ovules, pollen grains, seeds, scions, rootstocks, fruits, parts of fruits, flowers, roots, root tips, stems, hypocotyls, petioles, cotyledons, cuttings, anthers, pistils, stalks, meristems, protoplasts, cells, and/or the like, and portions thereof, isolated from ‘ML3326’ melon plants. In certain embodiments, the present disclosure provides pollen grains or ovules isolated from ‘ML3326’ melon plants. In some embodiment, the present disclosure provides protoplasts produced from ‘ML3326’ melon plants or parts. In some embodiment, the present disclosure provides tissue and/or culture produced from protoplasts or regenerable cells of and/or derived from ‘ML3326’ melon plants. In some embodiment, said protoplasts or regenerable are produced from a plant part selected from the group consisting of root, root tip, meristematic cell, stem, hypocotyl, petiole, cotyledon, leaf, flower, anther, pollen, pistil, stalk, and fruit. The present disclosure provides melon plants regenerated from tissue culture, where the plants regenerated from the tissue culture have all, or essentially all, of the physiological and morphological characteristics of ‘ML3326’ melon.

In some embodiment, the present disclosure provides a method of producing a seed of a ‘ML3326’-derived melon plant, including the steps: crossing a melon designated as ‘ML3326’, representative sample of seed having been deposited under NCIMB Accession Number X2, with itself or a second melon plant; and whereby seed of a ‘ML3326’-derived melon plant forms. In some embodiment, the method further includes (a) crossing a plant grown from ‘ML3326’-derived melon seed with itself or a second melon plant to yield additional ‘ML3326’-derived melon seed; (b) growing the additional ‘ML3326’-derived melon seed of step (a) to yield additional ‘ML3326’-derived melon plants; and (c) repeating the crossing and growing of steps (a) and (b) for at least one additional generation (e.g., an additional 3-10 generations) to generate further ‘ML3326’-derived melon plants. In some embodiment, the present disclosure further provides melon plants, melon parts from the melon plants, and seeds produced therefrom where the melon plant is grown from seed produced by the methods of any of the above embodiments.

In some embodiment, the present disclosure provides methods of vegetatively propagating a plant of melon variety ‘ML3326’, the method including the steps of: (a) collecting a tissue capable of being propagated from a plant of melon variety ‘ML3326’; and (b) producing a rooted plant from said tissue.

The present disclosure provides, among other things, a melon plant designated ‘ML3326’. The present disclosure thus relates to the plants of melon ‘ML3326’, the plant parts of melon ‘ML3326’, and to the seeds of melon ‘ML3326’. The present disclosure also relates to methods for producing other melon cultivars or hybrids derived from melon ‘ML3326’ and to the melon cultivars and hybrids derived by the use of those methods.

In some embodiment, the present disclosure provides a method of producing a plant of melon variety ‘ML3326’ comprising an added desired trait (e.g., a trait that is advantageous for commercialization, production, and/or marketability), the method comprising introducing a transgene conferring the desired trait into a plant of melon variety ‘ML3326’.

In some embodiment, the present disclosure provides single gene converted plants of melon ‘ML3326’. The single transferred gene may preferably be a dominant or recessive allele. In various embodiments, the single transferred gene can confer a trait such as sex determination, herbicide resistance, insect resistance, resistance for bacterial, fungal, or viral disease, improved harvest characteristics, enhanced nutritional quality, and/or improved agronomic quality. The single gene may be a naturally occurring melon gene or a transgene introduced through genetic engineering techniques.

In some embodiment, the present disclosure provides methods for developing melon plants in a melon plant breeding program using plant breeding techniques such as recurrent selection, backcrossing, pedigree breeding, restriction fragment length polymorphism enhanced selection, genetic marker enhanced selection, and/or transformation. The present disclosure further includes plants and plant parts (e.g., seeds) produced by such breeding methods.

In some embodiments, the present disclosure provides melon seeds produced by methods of making a melon variety of the present disclosure. In some embodiments, the present disclosure provides melon plants, and/or parts thereof, obtained from growing seeds of the present disclosure. In some embodiments, the present disclosure provides melon plants, and/or parts thereof, having all, or essentially all, of the physiological and morphological characteristics of a ‘ML3627’ or ‘ML3326’ melon plant of the present disclosure. In some embodiments, the present disclosure provides melon tissue culture, obtained from or produced using plants or plant parts of the present disclosure. In some embodiments, a tissue culture of the present disclosure is produced from a plant part selected from the group consisting of a leaf, anther, pistil, stem, petiole, root, root tip, fruit, seed, flower, cotyledon, hypocotyl, embryo, and meristematic cell.

In some embodiment, the present disclosure provides a method of producing a melon fruit, said method comprising growing the melon plant described herein until it sets at least one fruit, and harvesting the fruit.

In addition to the exemplary aspects and embodiments described above, further aspects and embodiments will be understood from the following descriptions.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows cross sections of whole fruits at maturity of melon variety ‘ML3627’.

FIG. 2 shows cross sections of whole fruits at maturity of melon variety ‘ML3326’.

### DETAILED DESCRIPTION

The present disclosure provides melon varieties exhibiting desired characteristics (e.g., characteristics that are advantageous for commercialization, production, and/or marketability). These desired characteristics can relate to, e.g., flesh taste (e.g., sweetness), flesh color, flesh texture, fruit size, fruit shape, fruit rind pattern, fruit shelf life, fruit yield, disease resistance, pest (e.g., insect) resistance, drought tolerance, flood tolerance, heat tolerance, cold tolerance, herbicide resistance, modified fatty acid metabolism, modified carbohydrate metabolism, nutritional quality, sex determination, male sterility, harvestability, and/or industrial utility).

There are numerous steps in the development of any novel, desirable plant germplasm. Plant breeding can begin with the identification of a desired characteristic or set of characteristics, establishment of program goals, and definition of specific breeding objectives.

The next step can be selection of germplasm that possess traits useful for development of varieties that will meet the program goals. The selected germplasm can then be crossed to recombine the desired traits (e.g., traits that are advantageous for commercialization, production, and/or marketability), and then developed into varieties or parent lines by breeding and selection. Breeding programs can produce a variety or hybrid that provides desired or improved combination of traits.

Choice of breeding or selection methods can depend on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and/or recurrent selection.

The complexity of inheritance influences choice of the breeding method. Backcross breeding is used to transfer one or a few favorable genes for a highly heritable trait into a commercially desirable cultivar. This approach has been used extensively for breeding disease-resistant varieties. Various recurrent selection techniques can be used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program may include a periodic, objective evaluation of the efficiency of the breeding procedure. Evaluation criteria vary depending on the goal and objectives, and can include gain from selection per year based on comparisons to an appropriate standard, overall value of the advanced breeding lines, and number of successful cultivars produced per unit of input (e.g., per year, per dollar expended, etc.).

Promising advanced breeding lines can be thoroughly tested and compared to appropriate standards in environments representative of the commercial target area(s) for at least three years. The best lines can then be candidates for new commercial cultivars. Those still deficient in a few traits may be used as parents to produce new populations for further selection. These processes, which lead to the final step of marketing and distribution, may take from ten to twenty years from the time the first cross or selection is made.

One goal of melon plant breeding is to develop new, unique, and genetically superior melon cultivars and hybrids. A breeder can initially select and cross two or more parental lines, followed by repeated selfing and selection, producing many new genetic combinations. A plant breeder can then select which germplasms to advance to the next generation. These germplasms may then be grown under different geographical, climatic, and soil conditions, and further selections can be made during, and at the end of, the growing season.

The development of commercial melon cultivars can include the development and/or selection of melon parental lines, the crossing of these lines, and the evaluation of the crosses.

Pedigree breeding and recurrent selection breeding methods may be used to develop cultivars from breeding populations. Breeding programs can be used to combine desirable traits from two or more varieties or various broad-based sources into breeding pools from which lines can be developed by selfing and selection of desired phenotypes (e.g., phenotypes that are advantageous for commercialization, production and/or marketability). The new lines can be crossed with other lines and the hybrids from these crosses can be evaluated to determine which have commercial potential.

Pedigree breeding is generally used for the improvement of self-pollinating crops or inbred lines of cross-pollinating crops. Two parents which possess favorable, complementary traits can be crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families can be selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines can be tested for potential release as new cultivars.

Mass and recurrent selections can be used to improve populations of either self-or cross-pollinating crops. A genetically variable population of heterozygous individuals is either identified or created by intercrossing several different parents. The best plants can be selected based on individual superiority, outstanding progeny, or excellent combining ability. The selected plants can be intercrossed to produce a new population in which further cycles of selection can be continued.

Backcross breeding may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent can be selected and repeatedly crossed (backcrossed) to the recurrent parent. The resulting plant is expected to have the attributes of the recurrent parent (e.g., cultivar) and the desirable trait transferred from the donor parent.

New varieties can also be developed from more than two parents. The technique, known as modified backcrossing, uses different recurrent parents during the backcrossing. Modified backcrossing may be used to replace the original recurrent parent with a variety having certain more desirable characteristics or multiple parents may be used to obtain different desirable characteristics from each.

In addition to being used to create a backcross conversion, backcrossing can also be used in combination with pedigree breeding. As discussed previously, backcrossing can be used to transfer one or more specifically desirable traits from one variety, the donor parent, to a developed variety called the recurrent parent, which has overall good agronomic characteristics yet lacks that desirable trait or traits. However, the same procedure can be used to move the progeny toward the genotype of the recurrent parent, but at the same time retain many components of the nonrecurrent parent by stopping the backcrossing at an early stage and proceeding with selfing and selection. For example, an inbred melon line may be crossed with another variety to produce a first generation progeny plant. The first generation progeny plant may then be backcrossed to one of its parent varieties to create a BC1 or BC2. Progeny can be selfed and selected so that the newly developed variety has many of the attributes of the recurrent parent and yet several of the desired attributes of the nonrecurrent parent. This approach leverages the value and strengths of the recurrent parent for use in new melon varieties.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines can be derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques known in the art that are available for the analysis, comparison and characterization of plant genotype. Such techniques include, without limitation, High Resolution Melting (HRM), DNA- or RNA-sequencing, CAPS Markers, ELISA, Western blot, microarrays, Single Nucleotide Polymorphisms (SNPs), Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Differential Display Polymerase Chain Reaction (DD-PCR), Quantitative Real-Time Polymerase Chain Reaction (qRT-PCR), DNA Amplification Fingerprinting (DAF), Sequence Characterized Amplified Regions (SCARs), Amplified Fragment Length Polymorphisms (AFLPs), and Simple Sequence Repeats (SSRs, which are also referred to as Microsatellites).

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding may also be used to introduce new traits into melon varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid, or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The production of double haploids can also be used for the development of homozygous varieties in a breeding program. Double haploids can be produced by the doubling of a set of chromosomes from a heterozygous plant to produce a completely homozygous individual. For example, see Wan et al., Theor. Genet Genet., 77:889-892, 1989.

Additional non-limiting examples of breeding methods that may be used include, without limitation, those found in Principles of Plant Breeding, John Wiley and Son, pp. 115-161, 1960; Allard, 1960; Simmonds, 1979; Sneep et al., 1979; Fehr, 1987; “Carrots and Related Vegetable Umbelliferae”, Rubatzky, V.E., al. al., 1999.

### DEFINITIONS

In the description that follows, a number of terms are used. In order to provide a clear and consistent understanding of the specification and claims, including the scope to be given such terms, the following definitions are provided:
Allele. The allele is any of one or more alternative forms of a gene, all of which relate to one trait or characteristic. In a diploid cell or organism, the two alleles of a given gene occupy corresponding loci on a pair of homologous chromosomes.
Andromonoecious plant. A plant having both staminate and perfect flowers on the same plant.
Backcrossing. Backcrossing is a process in which a breeder repeatedly crosses hybrid progeny back to one of the parents, for example, a first-generation hybrid F₁ with one of the parental genotype of the F₁ hybrid.
Essentially all the physiological and morphological characteristics. A plant having essentially all the physiological and morphological characteristics of another plant means that the plants share essentially all physiological and morphological characteristics identified herein, except, e.g., where applicable, with respect to characteristics derived from a converted gene that differs between the plants as a result backcrossing, mutation, or genetic engineering.
Fusarium wilt. Fusarium wilt of melon is a disease that can be caused by the fungus Fusarium oxysporum f. sp. melonis (Fom). Infected plants can wilt, have brown-streaked vascular tissue, and collapse.
Gene. As used herein, “gene” refers to a segment of nucleic acid. A gene can be introduced into a genome of a species, whether from a different species or from the same species, using transformation or various breeding methods.
Monoecious plant. A plant having separate staminate and pistillate flowers on the same plant.
Powdery mildew. Powdery mildew of melon is a disease that can be caused by fungi such as Podosphaera xanthii (Px) (Sphaerotheca fuliginea) and Golovinomyces cichoracearum (Gc) (Erysiphe cichoracearum [symbol] . Infected plants have white powdery fungal mass on leaf surfaces, petioles, and young stems, and fruits that are malformed, sunburned, and ripened prematurely.
Propagate. To “propagate” a plant means to reproduce the plant by means including, but not limited to, seeds, cuttings, divisions, tissue culture, embryo culture, or other in vitro methods.
Quantitative Trait Loci (QTL). Quantitative trait loci refer to genetic loci that control, to some degree, one or more numerically representable traits, which numerically representable traits are often with values distributed continuously or semi-continuously over a range.
Regeneration. Regeneration refers to the development of a plant from tissue culture.
Single gene converted. Single gene converted or conversion plant refers to plants which are developed by a plant breeding technique called backcrossing wherein essentially all of the desired physiological and morphological characteristics of an inbred are recovered in addition to the single gene transferred into the inbred via the backcrossing technique, genetic engineering, or mutation.
Transgene. A “transgene” is a gene taken or copied from one organism and inserted into another organism. A transgene may be a gene that is foreign to the receiving organism or it may be a modified version of a native, or endogenous, gene.
Vat gene. Vat gene in melon is a resistance gene that confers resistance to aphids (e.g., Aphis gossypii) and prevents transmission of viruses such as potyviruses to melon.

### Overview of the Variety ‘ML3627’

Melon variety ‘ML3627’ is utilized in the fruit production. Melon variety ‘ML3627’ is a diploid, monoecious Harper melon variety that produces circular shaped fruit having grey ground skin color with greenish hue and orange flesh. The cork formation on fruit has medium thickness and linear and netted pattern. ‘ML3627’ is suitable for cultivation in open field. ‘ML3627’ is useful for commercial fruit production and has medium to long shelf-life.

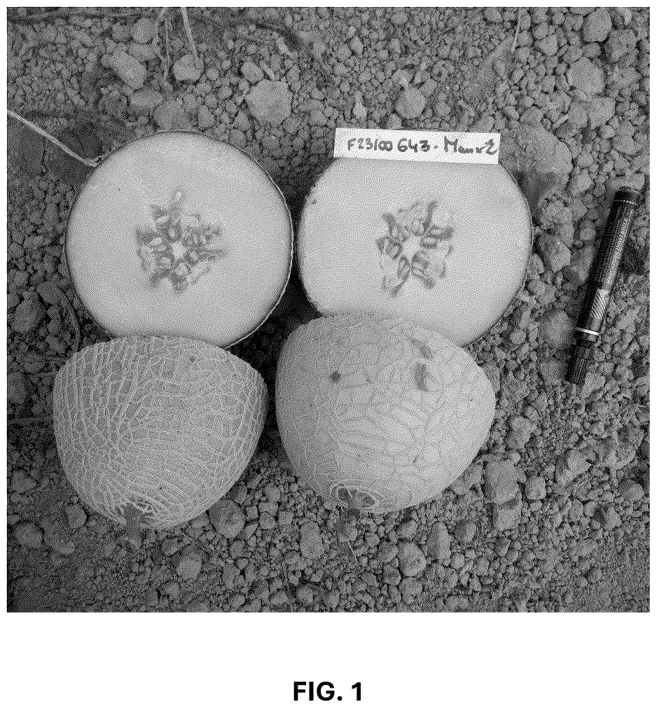

Additionally, melon variety ‘ML3627’ has resistance to Fusarium oxysporum f. sp. melonis (Fom) Races 0, 1, and 2, and intermediate resistance to Podosphaera xanthii (Px) (Sphaerotheca fuliginea) (Powdery mildew) Races 1, 2, 5, and 3-5 and Golovinomyces cichoracearum (Gc) (Erysiphe cichoracearum) (Powdery mildew) Race 1. ‘ML3627’ also has resistance to melon necrotic spot virus (MNSV) Strain 0 (MNSV: 0). FIG. 1 shows cross sections of whole fruits at maturity of melon variety ‘ML3627’. Melon variety ‘ML3627’ was chosen for, and/or is characterized by, resistance to Px Race 3-5, and medium to long shelf-life, and/or by a subset thereof (e.g., by a combination of two or more such characteristics that is desirable for production and/or marketability).

The variety has shown uniformity and stability for the traits, within the limits of environmental influence for the traits. No variant traits have been observed or are expected in ‘ML3627’.

### Objective Description of the Variety ‘ml3627’

In various embodiments, melon variety ‘ML3627’ can be characterized by the following morphologic and other characteristics.
Horticultural group: Harper
Use: Fruit production
Inflorescence:
   Sex expression (at full flowering): Monoecious
Young Fruit:
   Color of skin: Greyish green
   Intensity of green color of skin: Medium
Fruit:
   Length: Medium to long
   Shape in longitudinal section: Circular
   Ground color of skin: Grey
   Hue of ground color of skin: Greenish
   Density of dots: Absent or very sparse
   Density of patches: Absent or very sparse
   Grooves: Absent or very weakly expressed
   Creasing of surface: Absent or very weak
   Cork formation: Present
   Thickness of pattern of cork formation: Medium
   Pattern of cork formation: Linear and netted
   Density of pattern of cork formation: Medium
   Main color of flesh: Orange
Seeds:
   Length: Medium
   Shape: Not pine nut shape
   Color: Cream yellow
Shelf life of fruit: Medium to long
Growth conditions:
   Type of culture: Open field
Disease and stress resistance:
   Fusarium oxysporum f. sp. melonis (Fom) Races 0, 1, and 2: Present
   Fusarium oxysporum f. sp. melonis (Fom) Race 1-2: Absent
   Podosphaera xanthii (Px) (Sphaerotheca fuliginea) (Powdery mildew) Races 1, 2, 5, and 3-5: Intermediate resistant
   Golovinomyces cichoracearum (Gc) (Erysiphe cichoracearum) (Powdery mildew) Race 1: Intermediate resistant
   Colonisation by Aphis gossypii: Absent
   Melon necrotic spot virus (MNSV) Strain 0 (MNSV: 0): Present

### Overview of the Variety ‘ML3326’

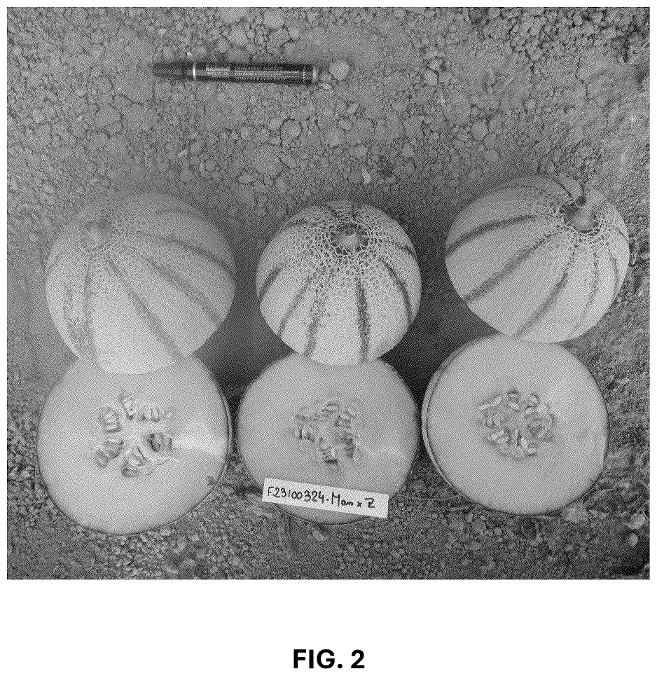

Melon variety ‘ML3326’ is utilized in the fruit production. Melon variety ‘ML3326’ is a diploid monoecious Harper melon variety that produces broad elliptic shaped fruit having grey ground skin color with greenish hue and orange flesh. The cork formation on fruit has medium thickness, strongly expressed grooves, and linear and netted pattern. ‘ML3326’ is suitable for cultivation in open field. ‘ML3326’ is useful for commercial fruit production and has medium to long shelf-life. Additionally, melon variety ‘ML3326’ has resistance to Fusarium oxysporum f. sp. melonis (Fom) Races 0, 1, and 2, and intermediate resistance to Podosphaera xanthii (Px) (Sphaerotheca fuliginea) (Powdery mildew) Races 1 and 2. FIG. 2 shows cross sections of whole fruits at maturity of melon variety ‘ML3326’. Melon variety ‘ML3326’ was chosen for, and/or is characterized by the pest resistance described and medium to long shelf-life, and/or by a subset thereof (e.g., by a combination of two or more such characteristics that is desirable for production and/or marketability).

The variety has shown uniformity and stability for the traits, within the limits of environmental influence for the traits. No variant traits have been observed or are expected in ‘ML3326’.

### Objective Description of the Variety ‘ML3326’

In various embodiments, melon variety ‘ML3326’ can be characterized by the following morphologic and other characteristics.
Horticultural group: Green Easter Shipper-Italian Cantaloupe
Use: Fruit production
Inflorescence:
   Sex expression (at full flowering): Monoecious
Young Fruit:
   Color of skin: Greyish green
   Intensity of green color of skin: Light to medium
Fruit:
   Length: Medium to long
   Shape in longitudinal section: Broad elliptic
   Ground color of skin: Grey
   Hue of ground color of skin: Greenish
   Density of dots: Absent or very sparse
   Density of patches: Absent or very sparse
   Grooves: Strongly expressed
   Depth of grooves: Medium
   Creasing of surface: Absent or very weak
   Cork formation: Present
   Thickness of pattern of cork formation: Medium
   Pattern of cork formation: Linear and netted
   Density of pattern of cork formation: Medium to dense
   Main color of flesh: Orange Seeds:
   Length: Medium
   Shape: Not pine nut shape
   Color: Cream yellow
Shelf life of fruit: Medium to long
Growth conditions:
   Type of culture: Open field
Disease and stress resistance:
   Fusarium oxysporum f. sp. melonis (Fom) Races 0, 1, and 2: Present
   Fusarium oxysporum f. sp. melonis (Fom) Race 1-2: Absent
   Podosphaera xanthii (Px) (Sphaerotheca fuliginea) (Powdery mildew) Races 1 and 2: Intermediate resistant
   Podosphaera xanthii (Px) (Sphaerotheca fuliginea) (Powdery mildew) Races 5 and 3-5: Susceptible
   Golovinomyces cichoracearum (Gc) (Erysiphe cichoracearum) (Powdery mildew) Race 1: Susceptible
   Colonisation by Aphis gossypii: Absent
   Melon necrotic spot virus (MNSV) Strain 0 (MNSV: 0): Absent

### Further Embodiments

The present disclosure is also directed to methods for producing a melon plant by crossing a first parent melon plant with a second parent melon plant, wherein the first or second melon plant is the melon plant ‘ML3627’ or ‘ML3326’. Further, both first and second parent melon plants may be ‘ML3627’ or ‘ML3326’. Therefore, any methods using melon ‘ML3627’ or ‘ML3326’ are part of the present disclosure: selfing, backcrosses, hybrid breeding, and crosses to populations. Any plants produced using melon ‘ML3627’ or ‘ML3326’ as at least one parent are within the scope of the present disclosure. Plants derived from melon ‘ML3627’ or ‘ML3326’ may be used, in certain embodiments, for the development of new melon varieties. By selecting plants having one or more desirable traits (e.g., traits that are advantageous for commercialization, production, and/or marketability), a plant derived from melon ‘ML3627’ or ‘ML3326’ is obtained which possesses some of the desirable traits of the hybrid as well as potentially other selected traits.

Melon varieties may be developed and selected for superior qualities including improved flesh taste (e.g., sweetness), flesh color, flesh texture, fruit size, fruit shape, fruit rind pattern, fruit shelf life, fruit yield, disease resistance, pest (e.g., insect) resistance, drought tolerance, flood tolerance, heat tolerance, cold tolerance, herbicide resistance, modified fatty acid metabolism, modified carbohydrate metabolism, nutritional quality, sex determination, male sterility, harvestability, and/or industrial utility.

The development of new varieties using one or more starting varieties is well known in the art. In accordance with this disclosure, novel varieties may be created by crossing melon ‘ML3627’ or ‘ML3326’ followed by multiple generations of breeding according to such well known methods. New varieties may be created by crossing with any second plant. In selecting such a second plant to cross for the purpose of developing novel lines, it may be desired to choose those plants which either themselves exhibit one or more selected desirable characteristics (e.g., characteristics that are advantageous for commercialization, production, and/or marketability) or which exhibit the desired characteristic(s) when in hybrid combination. Once initial crosses have been made, inbreeding and selection take place to produce new varieties. For development of a uniform line, often five or more generations of selfing and selection are involved.

It is preferred to breed for a combination of desirable plant characteristics and resistances to create a single variety or hybrid containing an improved combination of desirable traits from the parental germplasm. The development of commercial melon hybrids relates to the development of melon parental lines, the crossing of these lines, and the evaluation of the crosses. Hybrid varieties offer multiple advantages, including a combination of desirable dominant and recessive traits from a set of inbred parents. Pedigree breeding and recurrent selection breeding methods are used to develop cultivars from breeding populations. Breeding programs combine desirable traits from two or more varieties or various broad-based sources into breeding pools from which lines are developed by selfing and selection of desired phenotypes. The new lines are crossed with other lines and the hybrids from these crosses are evaluated to determine which have the desirable characteristics.

### Genetic Marker Profile Through SSR and First Generation Progeny

In addition to phenotypic observations, a plant can also be identified by its genotype. The genotype of a plant can be characterized through a genetic marker profile which can identify plants of the same variety or a related variety or be used to determine or validate a pedigree.

There are many further laboratory-based techniques known in the art that are available for the analysis, comparison and characterization of plant genotype. Such techniques include, without limitation, High Resolution Melting (HRM), DNA-or RNA-sequencing, CAPS Markers, ELISA, Western blot, microarrays, Single Nucleotide Polymorphisms (SNPs), Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Differential Display Polymerase Chain Reaction (DD-PCR), Quantitative Real-Time Polymerase Chain Reaction (qRT-PCR), DNA Amplification Fingerprinting (DAF), Sequence Characterized Amplified Regions (SCARs), Amplified Fragment Length Polymorphisms (AFLPs), and Simple Sequence Repeats (SSRs, which are also referred to as Microsatellites).

Particular markers used for these purposes are not limited to any particular set of markers, but are envisioned to include any type of marker and marker profile which provides a means of distinguishing varieties.

The present disclosure includes a melon plant characterized by molecular and physiological data obtained from the representative sample of said variety deposited with the National Collection of Industrial, Food and Marine Bacteria Ltd. (NCIMB Ltd.). Further provided by the disclosure is a melon plant formed by the combination of the disclosed melon plant or plant cell with another melon plant or cell and including the homozygous alleles of the variety.

Means of performing genetic marker profiles using SSR polymorphisms are well known in the art. SSRs are genetic markers based on polymorphisms in repeated nucleotide sequences, such as microsatellites. A marker system based on SSRs can be highly informative in linkage analysis relative to other marker systems in that multiple alleles may be present. Another advantage of this type of marker is that, through use of flanking primers, detection of SSRs can be achieved, for example, by polymerase chain reaction (PCR), thereby eliminating the need for labor-intensive Southern hybridization. PCR detection is done by use of two oligonucleotide primers flanking the polymorphic segment of repetitive DNA. Repeated cycles of heat denaturation of the DNA followed by annealing of the primers to their complementary sequences at low temperatures, and extension of the annealed primers with DNA polymerase, include the major part of the methodology.

Following amplification, markers can be scored by electrophoresis of the amplification products. Scoring of marker genotype is based on the size of the amplified fragment, which may be measured by the number of base pairs of the fragment. While variation in the primer used or in laboratory procedures can affect the reported fragment size, relative values should remain constant regardless of the specific primer or laboratory used. When comparing varieties it is preferable if all SSR profiles are performed in the same lab.

### Single-Gene Conversions

When the terms melon plant, cultivar, hybrid, or melon line are used in the context of the present disclosure, this also includes any single gene conversions of that line. The term “single gene converted plant” as used herein refers to those melon plants which are developed by a plant breeding technique called backcrossing wherein essentially all of the desired physiological and morphological characteristics (e.g., physiological and morphological characteristics advantageous for commercialization, production, and/or marketability) of a cultivar are recovered in addition to the single gene transferred into the line via the backcrossing technique. Backcrossing methods can be used with the present disclosure to improve or introduce a characteristic into the line. The term “backcrossing” as used herein refers to the repeated crossing of a hybrid progeny back to one of the parental melon plants for that line, backcrossing 1, 2, 3, 4, 5, 6, 7, 8, or more times to the recurrent parent. The parental melon plant which contributes the gene for the desired characteristic is termed the nonrecurrent or donor parent. This terminology refers to the fact that the nonrecurrent parent is used one time in the backcross protocol and therefore does not recur. The parental melon plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original cultivar of interest (recurrent parent) is crossed to a second line (nonrecurrent parent) that carries the single gene of interest to be transferred. The resulting progeny from this cross are then crossed again to the recurrent parent and the process is repeated until a melon plant is obtained wherein essentially all of the desired physiological and morphological characteristics of the recurrent parent are recovered in the converted plant, in addition to the single transferred gene from the nonrecurrent parent.

The selection of a suitable recurrent parent is an important step for a successful backcrossing procedure. The goal of a backcross protocol is to alter or substitute a single trait or characteristic in the original line. To accomplish this, a single gene of the recurrent cultivar is modified or substituted with the desired gene from the nonrecurrent parent, while retaining all, or essentially all, of the rest of the desired genetic traits of the original line, and thereby all, or essentially all, of the desired physiological and morphological characteristics of the original line. The choice of the particular nonrecurrent parent will depend on the purpose of the backcross; one of the major purposes is to add some commercially desirable, agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single gene traits have been identified that are not regularly selected for in the development of a new line but that can be improved by backcrossing techniques. Single gene traits may or may not be transgenic, and examples of these traits include but are not limited to, flesh taste (e.g., sweetness), flesh color, flesh texture, fruit size, fruit shape, fruit rind pattern, fruit shelf life, fruit yield, disease resistance, pest (e.g., insect) resistance, drought tolerance, flood tolerance, heat tolerance, cold tolerance, herbicide resistance, modified fatty acid metabolism, modified carbohydrate metabolism, nutritional quality, sex determination, male sterility, harvestability, and/or industrial utility. These genes are generally inherited through the nucleus. Several of these single gene traits are described in U.S. Pat. Nos. 5,777,196, 5,948,957 and 5,969,212, the disclosures of which are specifically hereby incorporated by reference.

### Tissue Culture

In some embodiments, reproduction of the variety can occur by tissue culture and regeneration. Tissue culture of various tissues of melon and regeneration of plants therefrom is well known and widely published. For example, reference may be had to Teng et al., HortScience. 1992, 27: 9,1030-1032, Teng et al., HortScience. 1993, 28: 6, 669-1671, Zhang et al., Journal of Genetics and Breeding. 1992, 46: 3, 287-290, Webb al. al., Plant Cell Tissue and Organ Culture. 1994, 38:1, 77-79, Curtis al. al., Journal of Experimental Botany. 1994, 45: 279,1441-1449, Nagata et al., Journal for the American Society for Horticultural Science. 2000,125: 6, 669-672, and Ibrahim et al., Plant Cell, Tissue and Organ Culture. (1992), 28(2): 139-145. It is clear from the literature that the state of the art is such that these methods of obtaining plants are routinely used and have a very high rate of success. Thus, another aspect of this disclosure is to provide cells which upon growth and differentiation produce melon plants having all, or essentially all, of the physiological and morphological characteristics of melon ‘ML3627’ or ‘ML3326’.

As used herein, the term “tissue culture” indicates a composition including isolated cells of the same or a different type or a collection of such cells organized into parts of a plant.

Exemplary types of tissue cultures are protoplasts, calli, meristematic cells, and regenerable plant cells that can generate tissue culture that are intact in plants or parts of plants, such as leaves, ovules, pollen grains, seeds, scions, rootstocks, fruits, parts of fruits, flowers, roots, root tips, stems, hypocotyls, petioles, cotyledons, anthers, pistils, stalks, and the like. Means for preparing and maintaining plant tissue culture are well known in the art. By way of example, a tissue culture including organs has been used to produce regenerated plants. The use of tissue culture to propagate melon plants is exemplified in Adelberg, J.W., B.B. Rhodes, Micropropagation from zygotic tissue of melon, C.E. Thomas (ed.) Proc. of the Cucurbitaceae 89: Evaluation and enhancement of cucurbit germplasm, Charleston S.C., USA; and al.ng et al., Shoot regeneration from immature cotyledon of melon, Cucurbit Genetics Coop. 17:111-115 (1994).

Tissue culture of melon can be used for the in vitro regeneration of melon plants.

Tissues cultures of various tissues of melon and regeneration of plants therefrom are well-known and published. By way of example, tissue cultures, some comprising organs to be used to produce regenerated plants via somatic embryogenesis, have been described in Debeaujon et al., Plant cell, Tissue and Organ Culture 34:91-100 (1993). Somatic embryogenesis and plant recovery are obtained from numerous sources including protoplasts, but the best results are observed with explants coming from seedlings, especially cotyledons and hypocotyls. It is clear from the literature that the state of the art is such that these methods of obtaining plants are “conventional”in the sense that they are routinely used and have a very high rate of success.

Thus, another aspect of this disclosure is to provide cells which upon growth and differentiation produce melon plants having all, or essentially all, of the physiological and morphological characteristics of melon ‘ML3627’ or ‘ML3326’.

Transformation of plant protoplasts also can be achieved using methods based on calcium phosphate precipitation, polyethylene glycol treatment, electroporation, and combinations of these treatments (see, e.g., Potrykus et al., 1985; Omirulleh al. al., 1993; Fromm et al., 1986; Uchimiya al. al., 1986; al.cotte et al., 1988). Transformation of plants and expression of foreign genetic elements is exemplified in Choi et al. (1994) and Ellul et al. (2003). Direct uptake of DNA into protoplasts using CaCl₂ precipitation, polyvinyl alcohol, or poly-L-ornithine has also been reported (see, e.g., Hain et al., 1985 and Draper al. al., 1982).

Electroporation of protoplasts and whole cells and tissues have also been described (see, e.g., Saker et al., 1998; Donn al. al., 1990; al.alluin et al., 1992; al. Laursen et al., 1994; Chupean et al., 1989).

### Additional Breeding Methods

This disclosure also provides methods for producing a melon plant by crossing a first parent melon plant with a second parent melon plant wherein the first or second parent melon plant is a melon plant of melon ‘ML3627’. Further, both first and second parent melon plants can come from melon ‘ML3627’. Thus, any such methods using melon ‘ML3627’ are part of the present disclosure: selfing, backcrosses, hybrid production, crosses to populations, and the like. All plants produced using melon ‘ML3627’ as at least one parent are within the scope of the present disclosure, including those developed from cultivars derived from melon melon ‘ML3627’. Advantageously, this melon cultivar could be used in crosses with other, different, melon plants to produce the first generation (F1) melon hybrid seeds and plants with superior characteristics. The cultivar of the disclosure can also be used for transformation where exogenous genes are introduced and expressed by the cultivar of the disclosure. Genetic variants of ‘ML3627’ plants and plant parts (e.g., seeds) are likewise encompassed herein, whether produced through traditional breeding methods or through techniques of genetic engineering (e.g., transformation of ‘ML3627’ by any of a number of protocols known to those of skill in the art).

The following describes breeding methods that may be used with melon ‘ML3627’ in the development of further melon plants. One such embodiment is a method for developing progeny melon plants in a melon plant breeding program comprising: obtaining the melon plant, or a part thereof, of ‘ML3627’, utilizing said plant or plant part as a source of breeding material; and selecting a melon ‘ML3627’ progeny plant with molecular markers in common with ‘ML3627’ and/or with physiological and/or morphological characteristics selected from the characteristics listed above. Breeding steps that may be used in the melon plant breeding program include pedigree breeding, backcrossing, mutation breeding, and recurrent selection. In conjunction with these steps, techniques such as RFLP-enhanced selection, genetic marker enhanced selection (for example, SSR markers) and the making of double haploids may be utilized.

Another method involves producing a population of melon ‘ML3627’ progeny melon plants, by crossing ‘ML3627’ with another melon plant, thereby producing a population of melon plants, which, on average, derive 50% of their alleles from melon ‘ML3627’. A plant of this population may be selected and repeatedly selfed or sibbed with a melon plant resulting from these successive filial generations. One embodiment of the present disclosure is the melon cultivar produced by this method and that has obtained at least 50% of its alleles from melon ‘ML3627’.

Progeny of melon ‘ML3627’ may also be characterized through their filial relationship with melon ‘ML3627’, as for example, being within a certain number of breeding crosses of melon ‘ML3627’. A breeding cross is a cross made to introduce new genetics into the progeny, and is distinguished from a cross, such as a self or a sib cross, made to select among existing genetic alleles. The lower the number of breeding crosses in the pedigree, the closer the relationship between melon ‘ML3627’ and its progeny. For example, progeny produced by the methods described herein may be within 1, 2, 3, 4, or 5 breeding crosses of melon ‘ML3627’.

The present disclosure also provides methods for producing a melon plant by crossing a first parent melon plant with a second parent melon plant wherein the first or second parent melon plant is a melon plant of ‘ML3326’. Further, both first and second parent melon plants can come from melon ‘ML3326’. Thus, any such methods using melon ‘ML3326’ are part of the present disclosure: selfing, backcrosses, hybrid production, crosses to populations, and the like. All plants produced using melon ‘ML3326’ as at least one parent are within the scope of the present disclosure, including those developed from cultivars derived from melon ‘ML3326’. Advantageously, this melon cultivar could be used in crosses with other, different, melon plants to produce the first generation (F1) melon hybrid seeds and plants with superior characteristics. The cultivar of the disclosure can also be used for transformation where exogenous genes are introduced and expressed by the cultivar of the disclosure. Genetic variants of ‘ML3326’ plants and plant parts (e.g., seeds) are likewise encompassed herein, whether produced through traditional breeding methods or through techniques of genetic engineering (e.g., transformation of ‘ML3326’ by any of a number of protocols known to those of skill in the art).

Another method involves developing melon plant progeny through a melon plant breeding program comprising: obtaining the melon plant, or a part thereof, of ‘ML3326’, utilizing said plant or plant part as a source of breeding material, and selecting a melon ‘ML3326’ progeny plant with molecular markers in common with ‘ML3326’ with physiological and/or morphological characteristics selected from the characteristics listed above. Breeding steps that may be used in the melon plant breeding program include pedigree breeding, backcrossing, mutation breeding, and recurrent selection. In conjunction with these steps, techniques such as RFLP-enhanced selection, genetic marker enhanced selection (for example, SSR markers) and the making of double haploids may be utilized.

Another method involves producing a population of melon ‘ML3326’ progeny melon plants, by crossing ‘ML3326’ with another melon plant, thereby producing a population of melon plants, which, on average, derive 50% of their alleles from melon ‘ML3326’. A plant of this population may be selected and repeatedly selfed or sibbed with a melon plant resulting from these successive filial generations. One embodiment of the present disclosure is the melon cultivar produced by this method and that has obtained at least 50% of its alleles from melon ‘ML3326’.

Progeny of melon ‘ML3326’ may also be characterized through their filial relationship with melon ‘ML3326’, as for example, being within a certain number of breeding crosses of melon ‘ML3326’. A breeding cross is a cross made to introduce new genetics into the progeny, and is distinguished from a cross, such as a self or a sib cross, made to select among existing genetic alleles. The lower the number of breeding crosses in the pedigree, the closer the relationship between melon ‘ML3326’ and its progeny. For example, progeny produced by the methods described herein may be within 1, 2, 3, 4, or 5 breeding crosses of melon ‘ML3326’.

Additional methods include, without limitation, chasing selfs. Chasing selfs involves identifying inbred plants among melon plants that have been grown from hybrid melon seed. Once the seed is planted, the inbred plants may be identified and selected due to their decreased vigor relative to the hybrid plants that grow from the hybrid seed. By locating the inbred plants, isolating them from the rest of the plants, and self-pollinating them (i.e., “chasing selfs”), a breeder can obtain an inbred line that is identical to an inbred parent used to produce the hybrid.

One of ordinary skill in the art of plant breeding would know how to evaluate the traits of two plant varieties to determine if there is no significant difference between the two traits expressed by those varieties. Thus the disclosure includes progeny plants of melon ‘ML3627’ or ‘ML3326’, comprising a combination of at least two variety traits selected from the group consisting of traits listed in the Summary of the Disclosure, so that said progeny melon plant is not significantly different for said traits than melon ‘ML3627’ or ‘ML3326’, respectively, as determined at the 5% significance level when grown in the same environmental conditions. Using techniques described herein, molecular markers may be used to identify said progeny plant as a melon ‘ML3627’ progeny plant or a melon ‘ML3326’ progeny plant. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as flesh taste (e.g., sweetness), flesh color, flesh texture, fruit size, fruit shape, fruit rind pattern, fruit shelf life, fruit yield, disease resistance, pest (e.g., insect) resistance, drought tolerance, flood tolerance, heat tolerance, cold tolerance, herbicide resistance, modified fatty acid metabolism, modified carbohydrate metabolism, nutritional quality, sex determination, male sterility, harvestability, and/or industrial utility.

The cultivar of the disclosure can also be used for transformation where exogenous genes are introduced and expressed by the cultivar of the disclosure. Genetic variants of ‘ML3627’ and ‘ML3326’ plants and plant parts (e.g., seeds) are likewise encompassed herein, whether produced through traditional breeding methods or through techniques of genetic engineering (e.g., transformation of ‘ML3627’ or ‘ML3326’ by any of a number of protocols known to those of skill in the art).

Mutations for use in mutation breeding can be induced in plants by using mutagenic chemicals such as ethyl methane sulfonate (EMS), by irradiation of plant material with gamma rays or fast neutrons, or by other means. The resulting nucleotide changes are random, but in a large collection of mutagenized plants the mutations in a gene of interest can be readily identified by using the TILLING (Targeting Induced Local Lesions IN Genomes) method (McCallum et al. (2000) Targeted screening for induced mutations. Nat. Biotechnol. 18, 455-457, and Henikoff et al. (2004) TILLING. Traditional mutagenesis meets functional genomics. Plant Physiol. 135, 630-636). The principle of this method is based on the PCR amplification of the gene of interest from genomic DNA of a large collection of mutagenized plants in the M2 generation. By DNA sequencing or by looking for point mutations using a single-strand specific nuclease, such as the CEL-I nuclease (Till et al. (2004) Mismatch cleavage by single-strand specific nucleases. Nucleic Acids Res. 32, 2632-2641), the individual plants that have a mutation in the gene of interest are identified. By screening many plants, a large collection of mutant alleles is obtained, each giving a different effect on gene expression or enzyme activity. The gene expression or protein levels can for example be tested by transcript analysis levels (e.g., by RT-PCR) or by quantification of protein levels with antibodies. Plants with the desired reduced gene expression or reduced protein expression are then back-crossed or crossed to other breeding lines to transfer only the desired new allele into the background of the crop wanted.

Genes of interest for use in breeding may also be edited using gene editing techniques including transcription activator-like effector nuclease (TALEN) gene editing techniques, clustered Regularly Interspaced Short Palindromic Repeat (CRISPR/Cas9) gene editing techniques, and/or zinc-finger nuclease (ZFN) gene editing techniques. For this, transgenic plants are generated expressing one or more constructs targeting the gene of interest. These constructs may include, without limitation, an anti-sense construct, an optimized small-RNA construct, an inverted repeat construct, a targeting construct, a guide RNA construct, a construct encoding a targeting protein, and/or a combined sense-anti-sense construct, and may work in conjunction with a nuclease, an endonuclease, and/or an enzyme, so as to downregulate the expression of a gene of interest.

As used herein, the term “plant” includes plant cells, plant protoplasts, plant cell tissue cultures from which melon plants can be regenerated, plant calli, plant clumps, and plant cells that are intact in plants or parts of plants, such as leaves, ovules, pollen grains, seeds, scions, rootstocks, fruits, parts of fruits, flowers, roots, root tips, stems, hypocotyls, petioles, cotyledons, anthers, pistils, stalks, and the like.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

### DEPOSIT INFORMATION

### Melon Variety ‘ML3627’

A deposit of at least 625 seeds of the melon variety ‘ML3627’ was made with the National Collection of Industrial, Food and Marine Bacteria Ltd. (NCIMB Ltd), Wellheads Place, Dyce, Aberdeen, AB21 7GB, United Kingdom, and assigned NCIMB Number X1. The seeds deposited with the NCIMB on [DATE] were obtained from the seed of the variety maintained by Enza Zaden USA, Inc., 7 Harris Place, Salinas, California 93901, United States since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the melon variety ‘ML3627’ will be maintained in the NCIMB, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

### Melon Variety ‘ML3326’

A deposit of at least 625 seeds of the melon variety ‘ML3326’ was made with the National Collection of Industrial, Food and Marine Bacteria Ltd. (NCIMB Ltd), Wellheads Place, Dyce, Aberdeen, AB21 7GB, United Kingdom, and assigned NCIMB Number X2. The seeds deposited with the NCIMB on [DATE] were obtained from the seed of the variety maintained by Enza Zaden USA, Inc., 7 Harris Place, Salinas, California 93901, United States since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the melon variety ‘ML3326’ will be maintained in the NCIMB, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

1. A melon seed selected from the group consisting of:
(a) a melon seed designated as ‘ML3627’, representative sample of seed having been deposited under NCIMB Accession Number X1; or
(b) a melon seed designated as ‘ML3326’, representative sample of seed having been deposited under NCIMB Accession Number X2.

2. A melon plant produced by growing the seed of claim 1.

3. A plant part from the plant of claim 2.

4. The plant part of claim 3, wherein said part is a leaf, an ovule, a pollen grain, a seed, a scion, a rootstock, a fruit, a flower, a root, a root tip, a stem, a hypocotyl, a petiole, a cotyledon, a cutting, an anther, a pistil, a stalk, a meristem, a protoplast, or a cell.

5. The plant part of claim 3, wherein said part is a fruit.

6. A melon plant having all or essentially all the physiological and morphological characteristics if the melon plant of claim 2.

7. A plant part from the plant of claim 6.

8. The plant part of claim 7, wherein said part is a leaf, an ovule, a pollen grain, a seed, a scion, a rootstock, a fruit, a flower, a root, a root tip, a stem, a hypocotyl, a petiole, a cotyledon, a cutting, an anther, a pistil, a stalk, a meristem, a protoplast, or a cell.

9. The plant part of claim 8, wherein said part is a fruit.

10. A pollen grain or an ovule of the plant of claim 2.

11. A protoplast produced from the plant of claim 2.

12. A tissue or cell culture produced from protoplasts or regenerable cells from the plant of claim 2.

13. The tissue or cell culture of claim 12, wherein said protoplasts or regenerable cells are produced from a plant part selected from the group consisting of root, root tip, meristematic cell, stem, hypocotyl, petiole, cotyledon, leaf, flower, anther, pollen, pistil, stalk, seed, embryo and fruit.

14. A melon plant regenerated from the tissue culture of claim 12, wherein:
(a) the plant has all, or essentially all, of the physiological and morphological characteristics of a melon plant produced by growing melon seed designated as ‘ML3627’, representative sample of seed having been deposited under NCIMB Accession Number X1; or
(b) the plant has all, or essentially all, of the physiological and morphological characteristics of a melon plant produced by growing melon seed designated as ‘ML3326’, representative sample of seed having been deposited under NCIMB Accession Number X2.

15. A method of producing a seed of a ‘ML3627’-derived melon plant, comprising:
crossing a melon designated as ‘ML3627’, representative sample of seed having been deposited under NCIMB Accession Number X1, with itself or a second melon plant;
whereby seed of a ‘ML3627’-derived melon plant forms.

16. The method of claim 15, further comprising:
(a) crossing a plant grown from ‘ML3627’-derived melon seed with itself or a second melon plant to yield additional ‘ML3627’-derived melon seed;
(b) growing the additional ‘ML3627’-derived melon seed of step (a) to yield additional ‘ML3627’-derived melon plants; and
(c) repeating the crossing and growing of steps (a) and (b) for at least one generation to generate further ‘ML3627’-derived melon plants.

17. The method of claim 16, wherein the at least one generation comprises 3-10 generations.

18. A method of producing a seed of a ‘ML3326’-derived melon plant, comprising:
crossing a melon designated as ‘ML3326’, representative sample of seed having been deposited under NCIMB Accession Number X2, with itself or a second melon plant;
whereby seed of a ‘ML3326’-derived melon plant forms.

19. The method of claim 18, further comprising:
(a) crossing a plant grown from ‘ML3326’-derived melon seed with itself or a second melon plant to yield additional ‘ML3326’-derived melon seed;
(b) growing the additional ‘ML3326’-derived melon seed of step (a) to yield additional ‘ML3326’-derived melon plants; and
(c) repeating the crossing and growing of steps (a) and (b) for at least one generation to generate further ‘ML3326’-derived melon plants.

20. The method of claim 19, wherein the at least one generation comprises 3-10 generations.

21. A method of producing a melon fruit, said method comprising growing the plant of claim 2 until it sets at least one fruit, and harvesting the fruit.

22. A method of vegetatively propagating a plant of melon variety ‘ML3627’, the method comprising the steps of:
(a) collecting a tissue capable of being propagated from a plant of melon variety ‘ML3627’, representative seed of said melon variety ‘ML3627’ having been deposited under NCIMB Accession Number X1; and
(b) producing a rooted plant from said tissue.

23. A method of vegetatively propagating a plant of melon variety ‘ML3326’, the method comprising the steps of:
(a) collecting a tissue capable of being propagated from a plant of melon variety ‘ML3326’, representative seed of said melon variety ‘ML3326’ having been deposited under NCIMB Accession Number X2; and
(b) producing a rooted plant from said tissue.
